(19) United States
(12) Patent Application Publication
(10) Pub. No.: US 2012/0053572 A1
(43) Pub. Date: Mar. 1, 2012

(54) INSTRUMENTS COATED WITH IRON OXIDE NANOPARTICLES FOR INVASIVE MEDICINE

(75) Inventors: Viorel Rusu, Eygelshoven (NL); Paul Borm, Meerssen (NL)

(73) Assignee: MagnaMedics GmbH, Aachen (DE)

(21) Appl. No.: 12/735,558

(22) PCT Filed: Jan. 27, 2009
(86) PCT No.: PCT/DE2009/000093

§ 371 Date: Jan. 3, 2011

(30) Foreign Application Priority Data
Jan. 28, 2008 (DE) 10 2008 006 402.5

(51) Int. Cl. A61B 17/00; B05D 3/00

(52) U.S. Cl. 606/1; 427/2.28

(57) ABSTRACT

Instruments coated with ferrofluids for invasive medicine can be imaged by magnetic resonance imaging (MRI) with high quality.

### CROSS-REFERENCE TO RELATED APPLICATIONS

This application is a National Stage application of International Application No. PCT/DE2009/000093, filed on Jan. 27, 2009, which claims priority of German application number 10 2008 006 402.5, filed on Jan. 28, 2008, both of which are incorporated herein by reference in their entireties.

### BACKGROUND OF THE INVENTION

1. Field of the Invention

The present invention relates to instruments used in invasive medicine and coated with ferrofluids. The instruments coated with ferrofluids are visible in magnetic resonance tomography (MRT).

2. Description of the Prior Art

Microtablets are known, for example, from DE 342 26 19 A1. The specification describes cylindrical shaped bodies having a convex upper side and under-side, the cylinder diameter and height of which are independently of one another in the range of from 1.0 to 2.5 mm and are in a ratio to one another of 1:0.5 to 1.5.

Nowadays, more than 20,000,000 magnetic resonance imaging (MRI) procedures are performed worldwide for various clinical indications. In light of the growing importance of the use of ionizing radiation in therapy and of the growing interest in minimally invasive therapies, it is unsurprising that magnetic resonance tomography has been slowly establishing itself in the area of radiology since about 1995. While magnetic resonance tomography was originally developed for diagnostic images, it is now used as a tool for performing and assessing minimally invasive therapeutic interventions. The relatively new field of use concerns areas such as intraoperative and endovascular MRI procedures. Minimally invasive endovascular procedures play an increasingly important role in the treatment of patients. For many reasons, the radiological procedures are attractive alternatives to surgical interventions and corresponding treatments. Examples of vascular treatments are balloon angioplasty, placement of stents or stent grafts, packing of an aneurysm, air embolism, and local delivery of medicaments.

A problem in MRT procedures is that the human or animal vessels, e.g. the diseased blood vessels, into which the endovascular instruments are inserted are made completely visible. In addition, it is useful that the position of the instruments can be located at an acceptable image frequency.

In medical diagnosis or therapy, methods that involve intervention in the body are referred to as invasive, for example a biopsy or a smear test. As an operating procedure that does not place too much strain on the patient, mention may be made of minimally invasive surgery.

Examples of instruments for invasive medicine are catheters, stents, pull wires and guide wires.

Catheters are hoses or tubes of various diameters which are made of plastic, latex, silicone or glass and with which hollow organs such as the bladder, stomach, intestines, vessels, etc., but also the ears and heart, can be explored, emptied, filled or flushed. This is done for diagnostic reasons (related to an examination) or for therapeutic reasons (related to a treatment).

Catheters can be used, for example, as
venous catheter: central venous catheter or indwelling venous cannula
in urology: catheters are used in urology to drain urine and as aids in diagnosis and therapy; in diagnosis, they serve to remove urine and to introduce medicaments and contrast agents
ureter catheter: draining urine from the kidney via the ureter into the bladder or to the outside
nephrostomy catheter: draining urine from the renal pelvis out through the skin
in cardiology: heart catheter
in hematology: port catheter
in anesthesia: peridural catheter
in ear, nose and throat surgery: Eustachian tube catheter
in dialysis therapy: peritoneal catheter for performing peritoneal dialysis.

In use, the instruments can, if appropriate, be introduced into the body through a tubular sleeve.

When introducing an instrument such as a catheter into the body, monitoring is necessary in order to control the introduction and to make the examination or the therapy visible.

Various methods are known by which instruments for invasive medicine in the human body are made visible.

From J. Magn. Resonance Imaging 23, 123 to 129 (2006), it is known to coat instruments for invasive medicine with paramagnetic particles. Dysprosium oxide is used as the paramagnetic material.

In Phys. Med. Biol 51 (2006) N127 to N137, various paramagnetic markers for coating instruments for invasive medicine are compared. Because of its higher susceptibility, dysprosium oxide is preferred to ferromagnetic and ferrimagnetic material.

WO 2005/110217 A1 describes how instruments for invasive medicine are coated with nanomagnetic material and imaged with the aid of magnetic resonance (MRT). The nanomagnetic materials used are films of FeAl, FeAlO and FeAlN.

WO 2005/120598 A1 describes a catheter guide wire which is provided with a contrast medium. The contrast medium used is iron powder having a grain size of below 10 μm.

WO 2007/000148 A2 describes rod-shaped bodies (e.g. instruments for minimally invasive interventions) which are composed of one or more filaments and of a non-ferromagnetic matrix material, which matrix material surrounds the filament or filaments or adheres them to one another and contains a dopant that generates magnetic resonance tomographic artefacts. Nanoparticles of rare earths are cited as dopant.

DE 10 2006 020 402 B3 discloses guide wires for microcatheters, which comprise diamond nanoparticles or ferrofluids suspended in liquid.

US 2005/0079132 A1 describes medical devices which, in order to make them visible in the magnetic field, contain nanomagnetic materials.

WO 2003/035161 A1 discloses medical devices made of polymer material which are visible in magnetic resonance tomography and which are coated with ferromagnetic material having a diameter of about 0.01 to about 50 μm.

WO 2003/099371 A1 discloses a guide wire for catheters with radiopaque markers encapsulated in its outer coating, for example gold, platinum or palladium. A hydrophilic coating can also be applied to the guide wire.

Document WO 2005/030286 A1 describes medical devices, for example stents, which are made visible in the magnetic field by having markers incorporated in them, for example steel particles.

US 2004/087933 A1 discloses a catheter guide wire which is formed from a solid core of continuous polymer material, preferably polyetheretherketone (PEEK), and was produced by extrusion. The guide wire narrows toward the distal end and can be provided with a hydrophilic coating.

DE 199 21 088 A1 describes implantable stents which are made of metallic and/or non-metallic material and which are provided with nanoscale particles that have a paramagnetic core and at least one shell absorbed on the core.

US 2006/249705 A1 discloses inorganic tubular structures, for example stents, which comprise nanomagnetic particles measuring less than 100 nm. The nanomagnetic particles are used to improve the visualization of the tubular structure in magnetic resonance tomography.

US 2005/107870 A1 discloses a medical instrument having a first coating of a bioactive material, which is located on at least part of the surface of the instrument, and a second coating layer, which comprises a polymer material and a nanomagnetic material, with the second layer being applied on the first layer.

Document US 2004/210289 A1 describes a composition comprising nanomagnetic particles which have a particle size of less than 100 nm and are made up of three different atoms.

US 2004/030379 A1 discloses medical instruments provided with a first coating, which comprises a bioactive substance, and with a second coating, which is applied on the first coating, with the second coating comprising a polymer material and magnetic particles. The magnetic particles are intended to be freed from their coating by application of a magnetic field in order to permit the release of the bioactive substance contained in the first coating.

US 2005/215874 A1 discloses a medical instrument comprising a biocompatible main body, which is provided with a marker for enhancing visibility in magnetic resonance tomography.

Patent specifications U.S. Pat. No. 4,989,608 and U.S. Pat. No. 5,154,179 disclose a catheter comprising a flexible tubular element in which ferromagnetic particles are embedded.

GB 2 182 451 discloses a method for generating NMR signals during imaging by magnetic resonance tomography, in which method the generation of NMR signals in a body part that is not to be imaged is prevented by the introduction of magnetic material that disturbs the magnetic field near it.

In practice, markers composed of dysprosium oxide are nowadays used to coat instruments for invasive medicine.

A disadvantage of the known systems is that their use in invasive medicine is possible only with the aid of materials that are not readily available and that are expensive, such as dysprosium oxide. The visualization of the instruments coated with dysprosium oxide is not entirely satisfactory in MRT.

### SUMMARY OF THE PRESENT INVENTION

The object of the present invention is to provide instruments for invasive medicine in which the markers are materials that are readily available and inexpensive, have no incompatibilities and permit high-quality visualization in MRT.

Instruments for invasive medicine were found which are coated with ferrofluids.

In the context of the present invention, ferrofluids are basically liquids that contain iron oxides. The ferrofluids are generally composed of small magnetic particles, which are suspended in a carrier liquid. Generally, the carrier liquid used is preferably a paint in which the ferrofluids form stable dispersions. The ferrofluids are present in solid and hardened form on the instruments.

In the context of the present invention, instruments for invasive medicine are preferred in which the iron oxide in the ferrofluids is present as nanoparticles.

In particular, instruments for invasive medicine are preferred wherein the iron oxide nanoparticles in the ferrofluids have an average diameter in the range of 10 to 1000 nm. Preferably, the iron oxide nanoparticles have an average diameter in the range of 100 to 300 nm, and particularly preferably in the range of 150 to 200 nm.

A particular embodiment of the present invention is wherein the iron oxide nanoparticles in the ferrofluids are substantially spherical.

A particular embodiment of the present invention is wherein the iron oxide nanoparticles are silanized.

A particular embodiment of the present invention is wherein the iron oxide particles are paramagnetic and are composed of FeO, Fe₂O₃, Fe₃O₄, mixed iron oxides, or mixtures of the iron oxides.

In particular, it is preferable that the nanoparticles in the ferrofluids are composed mainly of a (alpha) Fe₂O₃.

According to another particularly preferred embodiment, it is preferable that the nanoparticles in the ferrofluids are composed mainly of a (alpha) Fe₃O₄.

A particular embodiment of the present invention is wherein the ferrofluid is composed of a carrier liquid in which iron oxide nanoparticles are suspended.

In particular, it is preferable that the iron oxide particles in the ferrofluids are in colloidal suspension in the carrier liquid.

A particular embodiment of the present invention is wherein, in the ferrofluids, a dispersion of iron oxide nanoparticles is suspended in a carrier liquid.

A particular embodiment of the present invention is wherein, in the ferrofluids, a dispersion of iron oxide particles is suspended in an aprotic polar solvent in a carrier liquid.

Aprotic polar solvents can, for example, be tetrahydrofuran, dimethyl sulfoxide or dioxane.

A particular embodiment of the present invention is wherein, in the ferrofluids, the carrier liquid is a paint.

In the context of the present invention, preferred paints are, for example, polyurethanes, polyolefins, polyacrylates, polystyrenes, polyvinyl lactams and copolymers and mixtures of these components.

The paints can contain further customary components, such as solvents, which dry off after application.

Ferrofluids according to the present invention can contain iron oxide particles in a concentration in the range of 75 to 98% by weight, preferably in the range of 80 to 95% by weight, and particularly in the range of 85 to 90% by weight.

Ferrofluids are known per se (Physik in unserer Zeit, 32, 122 to 127 (2001)).

Preferred ferrofluids according to the present invention, wherein, in the ferrofluids, a dispersion of iron oxide nanoparticles is suspended in a carrier liquid, are novel.

Ferrofluids for the present invention can be produced by dispersing the iron oxide in an aprotic polar solvent and then suspending it in a manner known per se in the carrier liquid.

The suspensions of a dispersion of iron oxide particles in an aprotic polar solvent, which are obtained by adding the dispersion to a carrier liquid, generally contain iron oxide with a concentration in the range of 2 to 15% by weight, preferably in the range of 5 to 12% by weight, and in particular in the range of 8 to 10% by weight.

A particular embodiment of the present invention is wherein the ferrofluids wholly or partially cover the instruments for invasive medicine as a marking.

A particular embodiment of the present invention is wherein the instruments for invasive medicine are composed of a tubular or rod-shaped matrix material, which itself is not ferromagnetic and which is coated with a ferrofluid.

Matrix materials for instruments in invasive medicine that are used in magnetic resonance tomography can be all materials that are used in practice for these instruments. Examples that may be mentioned are plastics, latex and silicones.

A particular embodiment of the present invention is wherein the tubular matrix material forms a catheter or a stent.

A particular embodiment of the invention is wherein the matrix material forms a pull wire or guide wire.

A particular embodiment of the present invention is wherein the coating of the matrix material with the ferrofluid has a thickness in the range of about 10 to about 100 μm.

The proportion of iron oxide nanoparticles in the dried coating is preferably more than 20% by weight, particularly preferably more than 30% by weight, and very particularly preferably more than 65% by weight. The proportion of iron oxide nanoparticles in the dried coating is preferably not more than 80% by weight.

The present invention also relates to a method for producing instruments used in invasive medicine and coated with ferrofluids, which method comprises the matrix material being coated with the ferrofluid and the carrier liquid hardens.

A particular embodiment of the method according to the invention comprises the matrix material being coated with the ferrofluid by immersion, spraying or with an applicator (e.g. spin coating). Applicators can be, for example, brushes or spatulas (example: ink-jet method).

The ferrofluid can be applied to the instrument or to the shell thereof.

Another particular embodiment of the method according to the invention comprises solvents in the carrier liquid being removed by evaporation, if appropriate in a vacuum.

The method according to the invention can be performed as follows for example:

The medical instrument is immersed one or more times in the ferrofluid and then dried until hardening is complete.

### BRIEF DESCRIPTION OF THE DRAWINGS

FIG. 1 is a perspective view of the structure of instruments used in invasive medicine according to the present invention.

### DETAILED DESCRIPTION OF THE PRESENT INVENTION

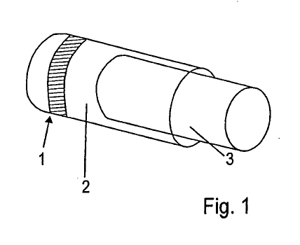

Referring to FIG. 1, the structure of instruments used in invasive medicine and coated with ferrofluids is described by way of example below:

A catheter 3 is guided in a tubular sleeve 2. The ferrofluid covers the sleeve 2 partially 1.

The present invention also relates to the use of instruments, coated with ferrofluids, for invasive medicine.

Here, the use of instruments, coated with ferrofluids, for visualization in MRT in invasive medical procedures is particularly preferred.

The visualization of catheters, stents, pull wires or guide wires in MRT is particularly preferred.

### Examples

### 1. Production of the Ferrofluid

Paramagnetic iron oxide nanoparticles, which are composed of α-Fe₃O₄ and/or Fe₂O₃ with a size in the range of 100 to 600 nm and which are spherical, are dispersed in an organic solvent such as tetrahydrofuran, dimethyl sulfoxide or dioxane. The dispersion usually contains between 10 and 30% by weight of the paramagnetic iron oxide nanoparticles. This dispersion is mixed with an adhesive coating polymer, for example a polyurethane, a polyolefin, a polyacrylate, a polystyrene, a polyvinyl lactam, and copolymers, or mixtures of these polymers and copolymers.

### 2. Coating of the Instrument for Invasive Medicine

2.1. The catheters are coated with a flexible polymer in which the marker positions are open and the rest has been covered. The coating is carried out by immersing the catheter in the ferrofluid according to Example 1.

The thickness of the coating on the catheter is in the range of 10 to 100 μm and can be controlled by the viscosity of the polymer-containing dispersion and/or by the number of immersions.

The size of the coating corresponds to the size of the uncovered surface on the catheter or to the size of the spring in the spin-coating method.

Further control is achieved by the concentration of the iron oxide particles in the ferrofluids.

Typical immersion times are in the range of one to two minutes, and the catheter should be allowed to dry for one to two minutes between the individual immersions.

The remainder of the drying takes place at room temperature for about eight hours.

After the drying, the organic solvent is evaporated and a hardened, stable coating with the ferrofluid remains on the catheter. There is a firm union between the flexible polymer and the coating.

The iron oxide nanoparticles are incorporated into the coating. To prevent migration of iron oxide particles and/or to facilitate the intravascular insertion of the instrument into the vessel, the coated instrument can be covered again with a biocompatible polymer (e.g. 0.2% chitosan in 1% strength acetic acid/0.1% strength polyacrylic acid) or alternatively with a hydrophilic coating.

2.2. Pull wires are needed in order to bring catheters or implants to the desired location during an operation or an examination.

Materials made of polyvinyl chloride, polyurethane, polyethylene ketone, polyethylene or nylon in combination with fibers or nanomaterials can be used as pull wires. The pull wires are coated in the same way as the catheters.

### 3. Visualization of the Coated Instrument for Invasive Medicine by MRI

Instruments used in invasive medicine are made visible by coating them with ferrofluids. The markers can be applied in various patterns on the instruments in order to facilitate use. During use, it is at all times possible to trace and locate the instrument. The instrument is traced and located electronically, if appropriate under magnification, on a screen. A ten-times magnification, for example, is possible without loss of image quality.

The markers meet the following conditions in the field of intervention:
They are biocompatible and safe.
They are small and are easy to apply to the instruments in question, without adversely affecting the use of the instruments.
They can be visualized with sharp definition in MRT and permit good differentiation between the tissue and the instrument.
They can be used at various field strengths in MRI.
They are passive, and no components are released.

### Illustrative Embodiments

### 1. Production of the Ferrofluids

Suspension 1: 30% by weight BAYFERROX® 318 in tetrahydrofuran (THF)
Suspension 2: Basecoat (a polyurethane-containing paint)

The product available from Lanxess under the trade name BAYFERROX® 318 or BAYFERROX® 318 M (the micronized version of BAYFERROX® 318) comprises spherical iron oxide nanoparticles having a diameter of 200 nm and having a core of Fe₃O₄ which makes up at least 90% by weight of the nanoparticles, and which has a shell made of SiO₂, which makes up about 3 to 5% by weight of the nanoparticles. The remaining approximately 5% by weight are accounted for by the residual moisture contained in the commercially available product. The density of these iron oxide nanoparticles is 4.6 g/cm³.

Various coating compositions were produced which differed from one another in terms of their content of iron oxide nanoparticles, by means of suspension 1 and suspension 2 first of all being mixed together in a ratio of 1:2, relative to % by weight, in order to obtain the coating composition M-12. The coating composition M-12 was then mixed with suspension 2 in the ratio 1:1, relative to % by weight, in order to obtain coating composition M-11. Moreover, coating composition M-11 was mixed with suspension 2 in the ratio 1:1, relative to % by weight, in order to obtain coating composition M-10. Some properties of the coating compositions are listed in Table 1. The proportion of iron oxide nanoparticles in the dried coating was determined after coating of the guide wires.

TABLE 1
Properties of the coating compositions

| | | | | Proportion of |
|---|---|---|---|---|
| | | Content | Content of | iron oxide nano- |
| | | of nano- | nano-particles | particles in dried |
| Coating | Density | particles | (number/100 | coating (% by |
| composition | (g/cm³) | (g/100 ml) | ml) | weight) |
| Suspension 2 | 0.876 | — | — | — |
| M-10 | 0.914 | 2.5 | 16.25 × 10¹⁵ | 12.8 ± 5 |
| M-11 | 0.946 | 5.0 | 32.5 × 10¹⁵ | 31.3 ± 5 |
| M-12 | 0.998 | 10.0 | 65 × 10¹⁵ | 62.0 ± 5 |

### 2. Coating of Catheter Guide Wires

Wires made of polyvinyl chloride (PVC) with a diameter of 2 mm and a length of 1 meter were obtained from Profilplast (Sittard, NL). Catheter guide wires with a core of polyetheretherketone (PEEK) and with a sheath of polyurethane were obtained from Biotronik AG (Bülach, CH).

Before being coated, the wires were cleaned with 70% by volume of isopropanol (in water). With the tip of a graphite lead that had been dipped immediately before into the respective coating composition, the wire to be coated was touched and then turned such that an annular visible band extending transversely with respect to the longitudinal axis of the wire was obtained. In this way, several bands were applied on each wire at a predetermined distance from one another.

The coated wires were dried for 24 hours at room temperature before their surface was cleaned by careful rubbing with distilled water.

### 3. Visibility of the Coated Guide Wires in MRT

The coated PVC wires were tested in a MnCl₂ solution (T1/T2=1030/140 ms at 1.5 T) with the aid of a 1.5 Tesla magnetic resonance tomograph and using the “FE tracking sequence PassTrack”.

In this test, all the coated wires were visible in MRI. The wires coated with M-10 gave the best results, that is to say the clearest images of the markers. The wires coated with M-12 gave very dark images. The concentration of the iron oxide nanoparticles was evidently too high to give clear images in this test set-up.

The usefulness of coated PEEK guide wires was tested using a 1.5 T whole-body tomograph (ESPREE®, Siemens Medical Solutions, Erlangen, Del.), which was equipped with a high-performance gradient system (gradient strength: 33 mT/m; pivot rate: 100 T/m/s). The renal arteries and the vena cava were explored by two wires. The exploration with the guide wires was monitored in real time. The visibility of the guide wires, their mobility and their steerability were assessed by the radiologist. The visibility of the guide wires coated with coating composition M-12 was excellent. The mobility and steerability of the guide wires was good and at least equivalent to a commercially available standard (Terumo Glidewire Stiff and Standard).

### 4. Comparison of Different Marking Materials

To be able to assess the quality of the visibility of PEEK guide wires coated with M-12, comparison tests were carried out in which PEEK guide wires were coated with different magnetic materials and were examined in an in vitro test on aorta phantoms.

For this purpose, the magnetic materials indicated in Table 2 were suspended in THF at the same concentration as for the production of the coating composition M-12 (Example 1). The guide wires were coated in the manner described in Example 2.

TABLE 2
Overview of the metallic nanoparticles used in the comparison test

| | Magnetic material | Source |
|---|---|---|
| 1 | Mixture of maghemite and magnetite | Dry MagFerro (MagnaMedics |
| | Fe₂O₃; size range: 150-300 nm | ferrofluid), batch: MF08010801 |
| 2 | Gadolinium-III oxide, nanoparticles, | Aldrich, 637335-10G |
| | 10-100 nm, 99.9% | |
| 3 | Fe₃O₄ | BAYFERROX ® 318 M, Lanxess |
| 4 | Fe (II, III) oxide nanoparticles, | 99.95% Alfa Aesar 044120 |
| | 10-100 nm | |
| 5 | Fe III oxide, gamma, nanoparticles, | Alfa Aesar 039951 |
| | 10-100 nm, 99.95% | |
| 6 | Fe₂O₃ 200 nm, 96.0% | Bayoxide E 8707H, Lanxess |

In the comparison tests, it was found that specimen 3, which involved PEEK guide wires coated with coating composition M-12, gave the best contrast and the clearest image of the markers in magnetic resonance tomography, better than the other coatings with iron oxide nanoparticles. All the coatings with iron oxide nanoparticles gave better MRT images than the marking with the standard gadolinium-III oxide.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A method for producing instruments used in invasive medicine and coated with ferrofluids, the instruments having a matrix material, said method comprising the steps of:
suspending iron oxide nanoparticles in an aprotic solvent to form a suspension;
dispersing said suspension in a polymer-containing carrier liquid to form a ferrofluid;
wholly or partially coating the matrix material of the instruments with the ferrofluid; and
hardening the carrier liquid.

2. The method according to claim 1, wherein the iron oxide nanoparticles are composed mainly of iron oxides selected from the group consisting of FeO, Fe₂O₃, Fe₃O₄, mixed iron oxides, and mixtures of the iron oxides.

3. The method according to claim 1, wherein the iron oxide nanoparticles have a shell of SiO₂.

4. The method according to claim 1, wherein the iron oxide nanoparticles are substantially spherical.

5. The method according to claim 1, wherein the iron oxide nanoparticles have a diameter of 10 to 1000 nm.

6. The method according to claim 1, wherein the ferrofluid has a content of iron oxide nanoparticles in the range of 2 to 15% by weight.

7. The method according to claim 1, wherein the ferrofluid contains between 10×10¹⁵ and 70×10¹⁵ iron oxide nanoparticles, per 100 ml.

8. The method according to claim 1, wherein the aprotic solvent is an aprotic polar solvent.

9. The method according to claim 1, wherein the solvent is selected from the group of solvents consisting of solvents that comprises tetrahydrofuran and chloroform.

10. The method according to claim 1, wherein the polymer-containing carrier liquid is a paint.

11. The method according to claim 10, wherein the paint comprises a polymer.

12. The method according to claim 1, wherein the instruments for invasive medicine comprise a tubular or rod-shaped matrix material, said matrix material not being ferromagnetic.

13. The method according to claim 12, wherein the tubular matrix material forms a catheter, a stent or other instruments for minimally invasive interventions.

14. The method according to claim 12, wherein the rod-shaped matrix material forms a pull wire or guide wire or other instruments for minimally invasive interventions.

15. The method according to claim 1, wherein the matrix material comprises a material selected from the group consisting of a polymer, metal and glass.

16. The method according to claim 1, wherein the coating of the matrix material has a thickness in the range of 10 μm to 100 μm.

17. Instruments for invasive medicine, wherein said instruments are produced by a method according to claim 1.

18. Instruments for invasive medicine according to claim 17, wherein said instruments have a coating with iron oxide nanoparticles, comprising 20 to 70% by weight of iron oxide nanoparticles in the dried coating, and the iron oxide nanoparticles are selected from the group consisting of FeO, Fe₂O₃, Fe₃O₄, mixed iron oxides, and mixtures of the iron oxides.

19. Use of instruments according to claim 17 for invasive medicine.

20. Use of instruments according to claim 17 for visualization in MRT during invasive medical interventions.

21. The method according to claim 2, wherein the iron oxide nanoparticles are composed mainly of at least one iron oxide selected from the group consisting of alpha Fe₂O₃ and alpha Fe₃O₄.

22. The method according to claim 5, wherein the iron oxide nanoparticles have a diameter of 100 to 300 nm.

23. The method according to claim 22, wherein the iron oxide nanoparticles have a diameter in the range of 150 to 200 nm.

24. The method according to claim 6, wherein the ferrofluid has a content of iron oxide nanoparticles in the range of 5 to 12% by weight.

25. The method according to claim 24, wherein the ferrofluid has a content of iron oxide nanoparticles in the range of 8 to 10% by weight.

26. The method according to claim 7, wherein the ferrofluid contains between 30×10¹⁵ and 65×10¹⁵ iron oxide nanoparticles, per 100 ml.

27. The method according to claim 11, wherein the paint comprises a polymer selected from the group of polymers consisting of polyurethanes, polyolefins, polyacrylates, polystyrenes, polyvinyl lactams, and copolymers and mixtures of these polymers.

28. Instruments for invasive medicine according to claim 18, wherein said Fe₂O₃, is alpha Fe₂O₃, and said Fe₃O₄ is alpha Fe₃O₄.

29. Use of instruments according to claim 20 for visualization in MRT during minimally invasive interventions.
